(19) United States
(12) Patent Application Publication
(10) Pub. No.: US 2026/0217809 A1
(43) Pub. Date: Jul. 30, 2026

(54) Collagen Type XVIII Assay

(71) Applicant: Nordic Bioscience A/S, Herlev (DK)

(72) Inventors: Tina Manon-Jensen, Brøndby (DK); Morten Karsdal, København Ø (DK)

(73) Assignee: Nordic Bioscience A/S, Herlev (DK)

(21) Appl. No.: 19/643,418

(22) Filed: Apr. 9, 2026

Related U.S. Application Data
(63) Continuation-in-part of application No. 18/145,767, filed on Dec. 22, 2022, now abandoned.
Application No. 18/145,767 is a continuation of application No. 16/494,441, filed on Mar. 14, 2018, now abandoned.

(30) Foreign Application Priority Data
Mar. 16, 2017 (GB) 1704182.3

(51) Int. Cl. C07K 16/18; C07K 14/78; G01N 33/68

(52) U.S. Cl. CPC C07K 16/18; C07K 14/78; G01N 33/6887; C07K 2317/33; C07K 2317/34; G01N 2333/78

(57) ABSTRACT

The present invention relates to an antibody, wherein the antibody is specifically reactive with short isoform collagen type XVIII, but does not react with intermediate isoform collagen type XVIII or with long isoform collagen type XVIII. The invention also relates to the use of the antibody in a method of immunoassay for detecting or quantitating short isoform collagen type XVIII, where the method may be used to evaluate haemophilic disease.

### CROSS-REFERENCE TO RELATED APPLICATIONS

This is a continuation-in-part application under 35 U.S.C. § 120 of pending application U.S. Ser. No. 18/145,767, filed Dec. 22, 2022, which is a continuation application under 35 U.S.C. § 120 of U.S. Ser. No. 16/494,441, filed Sep. 16, 2019, now abandoned, which is a national stage application under 35 U.S.C. § 371 of international application PCT/EP2018/056320, filed Mar. 14, 2018, now abandoned, which claims priority to European Application No. 1704182.3, filed Mar. 16, 2017, now abandoned.

### INCORPORATION OF SEQUENCE LISTING

The Sequence Listing XML file, entitled D7697CCIPSEQ.xml, created on Mar. 31, 2026, and with a size of 21,000 bytes, is hereby incorporated by reference in its entirety and made a part of this application.

### BACKGROUND OF THE INVENTION

### Field of the Invention

The present invention relates to a method for detecting the short isoform of Collagen type XVIII, and the use of this method in evaluating haemophilic diseases.

### Description of the Related Art

Recurrent haemarthroses due to vascular ruptures is a major complication in haemophilia, contributing to progressive joint damage, which leads to haemophilic (HF) arthropathy. The medical need in the HF field to reduce bleeding incidents requires measurement of the annual bleeding rate (ABR) in haemophiliacs. Although a crude measure, the ABR is associated with HF arthropathy (1) but is also a key parameter in clinical trials, ensuring quantifiable benefits to patients (2-4). Endothelial cell impairment and matrix quality may be associated with joint bleeds and later the development of HF arthropathy.

Vascular rupture is associated with the quality and turnover of the basement membrane (BM) located directly underneath the endothelial cells. Extracellular matrix turnover is a central pathological feature in many diseases due to epithelial or endothelial cell damage. While the endothelial cell function is debated, no quantifiable methods are available for specifically quantifying the damage to the vascular endothelium, which, subsequent to bleeding, results in exposure of the BM underlying the endothelial cells.

Quantifying the BM proteins specific to the endothelial cells may therefore have particular relevance to endothelial cell stability and rupture in haemophiliacs.

Collagen IV, XV ad XVIII represent the most well-known collagens of the vascular BMs, responsible for maintaining vessel wall structure and integrity of the membrane (FIG. 1A) (5-7).

Type XVIII collagen exists in three isoforms: short, intermediate, and long, localized in various basement membrane zones (4-6) (FIG. 1B). All three isoforms contain a thrombospondin 1-like domain and 10 triple helical collagenous domains (Col1-19) flanked by 11 noncollagenous domains (NC1-11). The NC1 domain contains a C-terminal endostatin domain that has antiangiogenic properties (7). The short isoform is endothelial specific and is found in blood vessels and around muscular structures. Here, zero or only very low amounts of the intermediate and long isoforms are present (5). Following remodelling, damage, and degradation of the vascular BM the short isoform of collagen type XVIII may be affected and degraded, releasing measurable fragments of type XVIII collagen, as have been undertaken with other types of collagen (8,9).

Mutations in type XVIII collagen have also been lined to the autosomal recessive disorder noblock syndrome (KS). KS is characterized by various eye defects leading to blindness at a young age (11,12). Morever, col18a/−/− knock-out mice showed delayed regression of blood vessels in the vitreous along the surface of the retina, impaired angiogenesis of retinal vessels and altered iris BM structure (8, 13-16). Thus, collagen XVIII is essential for controlling blood vessel formation in the eye, and possibly an important component in the BM zones of the entire vascular system (17).

The present Applicant is unaware of any biomarkers and/or antibodies specific to the short isotype of type XVIII collagen. The majority of commercially available antibodies recognize the C-terminal endostatin end of type XVIII collagen hence it is not possible to differentiate between the three isotypes using those antibodies.

Thus, there is a need for antibodies and/or biomarkers measuring the specific short isotype of type XVIII collagen and excluding the two other isoforms to quantify vascular specific basement membrane turnover in terms of detection of endothelial type XVIII collagen content.

### SUMMARY OF THE INVENTION

The Applicant has now developed an assay for detecting the short isoform of collagen type XVIII, and has used that assay to assess the clinical relevance of turnover of collagen type XVIII in patients diagnosed with HF arthropathy.

In a first aspect, the invention relates to an antibody specifically reactive with short isoform collagen type XVIII, wherein said antibody does not react with intermediate isoform collagen type XVIII or with long isoform collagen type XVIII.

Preferably, the antibody is specifically reactive with an N-terminal epitope of short isoform collagen type XVIII. Preferably, the Np534minal epitope is an epitope that is exposed after cleavage and removal of the N-terminal signal peptide of short isoform collagen type XVIII. Preferably, the antibody is specifically reactive with an N-terminal epitope comprised in the N-terminal amino acid sequence of H₂N-EPERISEEVG . . . (SEQ ID NO: 1). Preferably, the antibody is specifically reactive with an N-terminal epitope comprising the N-terminal amino acid sequence H₂N-EPERIS . . . (SEQ ID NO: 2).

Preferably, the antibody does not specifically recognise or bind an N-extended elongated version of said N-terminal amino acid sequence which is H₂N-AEPERISEEVG . . . (SEQ ID NO: 3) and/or does not specifically recognise or bind an N-truncated version of said N-terminal amino acid sequence which is H₂N-PERISEEVG . . . (SEQ ID NO: 4).

The antibody may be a monoclonal or polyclonal antibody. Preferably, the antibody is a monoclaonal antibody.

A monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGI, IgG2, IgG3, and IgG4 in the case of humans and IgGI, IgG2a, IgG2b, IgG2c and IgG3 in the case of mice. The monoclonal antibody may preferably be of the IgG isotype, including any one of the IgG subclasses.

Antibodies can be generated from B cell clones as described in the examples. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

The monoclonal antibody may preferably comprise one or more complementarity-determining regions (CDRs) selected from:

CDR-H1:
(SEQ ID NO: 10)
TSGMSVG,
CDR-H2:
(SEQ ID NO: 11)
HIWWNDDKDYSPVLKS,
CDR-H3:
(SEQ ID NO: 12)
MVFYTSTSGFDV,
CDR-L1:
(SEQ ID NO: 13)
RSSQNIVHTNGNTYLE,
CDR-L2:
(SEQ ID NO: 14)
RVSNRFS,
and
CDR-L3:
(SEQ ID NO: 15)
FHASHVPYT.

Preferably the monoclonal antibody comprises at least 2, 3, 4, 5 or 6 of the above listed CDR sequences.

Preferably the monoclonal antibody has a light chain variable region comprising the CDR sequences

CDR-L1:
(SEQ ID NO: 13)
RSSQNIVHTNGNTYLE,
CDR-L2:
(SEQ ID NO: 14)
RVSNRFS
and
CDR-L3:
(SEQ ID NO: 15)
FHASHVPYT.

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 16)
RSSQNIVHTNGNTYLEWYLQKPGQSPKLLIYRVSNRFSGVPDRFSG
SGSGTDFTLKISRVEAEDLGVYYCFHASHVPYT.

Preferably the monoclonal antibody has a heavy chain variable region comprising the CDR sequences:

CDR-H1:
(SEQ ID NO: 10)
TSGMSVG,
CDR-H2:
(SEQ ID NO: 11)
HIWWNDDKDYSPVLKS
and
CDR-H3:
(SEQ ID NO: 12)
MVFYTSTSGFDV.

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 17)
TSGMSVGWIRQPSGRGLEWLAHIWWNDDKDYSPVLKSRLTISKDSS
NNQVFLKIASVVAADTATYYCARMVFYTSTSGFDV.

As used herein, the framework amino acid sequences between the CDRs of an antibody are substantially identical or substantially similar to the framework amino acid sequences between the CDRs of another antibody if they have at least 70%, 80%, 90% or at least 95% similarity or identity. The similar or identical amino acids may be contiguous or non-contiguous.

The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In certain preferred embodiments, the monoclonal antibody may comprise the light chain variable region sequence:

(SEQ ID NO: 18)
DVLMTQTPLSLPVSLGDQASISCRSSQNIVHTNGNTYLEWYLQKPG
QSPKLLIYRVSNRFSGVPDRFSGSGSGTDFTLKISRVEAEDLGVYY
CFHASHVPYTFGGGTKLEIK

and/or the heavy chain variable region sequence:

(SEQ ID NO: 19)
QVTLKESGPGILQPSQTLSLTCSFSGFSLSTSGMSVGWIRQPSGRG
LEWLAHIWWNDDKDYSPVLKSRLTISKDSSNNQVFLKIASVVAADT
ATYYCARMVFYTSTSGFDVWGAGTTVTVSS.

(CDRs bold and underlined; Framework sequences in italics)

In a second aspect, the invention relates to a method of immunoassay for detecting or quantitating in a sample short isoform collagen type XVIII, wherein said method comprises contacting a sample comprising said short isoform collagen type XVIII with an antibody as described supra, and determining the amount of binding of said antibody.

The present invention may be directed to a method of detecting short isoform collagen type XVIII in a human patient, said method comprising:
a. obtaining a sample from the human patient; and
b. detecting whether said short isoform collagen type XVIII is present in the sample by contacting the sample with an antibody (as described supra) and detecting binding between short isoform collagen type XVIII and the antibody.

Preferably, the method comprises detecting or quantitating an N-terminal epitope of short isoform collagen type XVIII. The N-terminal epitope is preferably comprised in the N-terminal amino acid sequence H₂N-EPERISEEVG . . . (SEQ ID NO: 1). Preferably, the N-terminal epitope comprises the N-terminal amino acid sequence H₂N-EPERIS . . . (SEQ ID NO: 2).

Preferably, the sample is a biofluid. The biofluid may be, but is not limited to, serum, plasma, urine, cerebrospinal fluid, or amniotic fluid.

The immunoassay may be a competition assay or a sandwich assay. The immunoassay may be a radioimmunoassay or an enzyme-linked immunosorbent assay.

The method may further comprise correlating the quantity of the short isoform collagen type XVIII determined by the method with standard haemophilic disease samples of known disease severity to evaluate the severity of a haemophilic disease. In this regard, “standard haemophilic disease samples” means samples obtained from subjects known to have a haemophilic disease of a known severity.

Alternatively, or in addition to, the method may comprise comparing the quantity of said short isoform collagen type XVIII determined by said method with standard values associated with healthy subjects to evaluate the presence and/or severity of a haemophilic disease. In this regard “standard values associated with healthy subjects” means standardised quantities of short isoform collagen type XVIII determined by the method described supra for subjects considered to be healthy, i.e., without a haemophilic disease. The standardisation will depend on the height, weight, gender, etc. of the healthy subject.

Alternatively, or in addition to, the method may further comprise quantifying the amount of collagen type XVIII in at least two samples obtained from a subject at a first time point and at at least one subsequent time point, wherein an increase in the quantity of collagen type XVIII from the first time point to the at least one subsequent time point is indicative of a deterioration in a haemophilic disease from the first time point to the at least one subsequent time point, or wherein a decrease in the quantity of collagen type XVIII from the first time point to the at least one subsequent time point is indicative of an improvement in a haemophilic disease from the first time point to the at least one subsequent time point.

The haemophilic disease may be haemophilic arthropathy. The method described supra may also be used to evaluate Knoblock syndrome.

In another aspect, the present invention is directed to a method for evaluating the efficacy of a drug for treating a haemophilic disease. The method comprises using the method as described above to quantify the amount of collagen type XVIII in at least two biological samples obtained from a subject at a first time point and at at least one subsequent time point during a period of administration of the drug to the subject. A reduction in the quantity of collagen type XVIII from the first time point to the at least one subsequent timpe point during the period of administration of the drug is indicative of an efficacious drug for treating a haemophilic disease.

In a final aspect, the invention relates to an assay kit for determining the quantity of short isoform collagen type XVIII, comprising an antibody as described supra and at least one of:
a streptavidin coated 96 well plate,
a peptide which is reactive with said antibody, which may be a biotinylated peptide H₂N-EPERISEEVG-L-Biotin (SEQ ID NO: 5), wherein L is an optional linker,
an optionally biotinylated secondary antibody for use in a sandwich immunoassay,
a calibrator peptide comprising the N-terminal sequence H₂N-EPERISEEVG . . . (SEQ ID NO: 6),
an antibody HRP labelling kit,
an antibody radiolabeling kit, or
an assay visualization kit.

### BRIEF DESCRIPTION OF THE FIGURES

FIG. 1A. Structure of the vascular BM. The capillary subendothelial layer is composed of a BM and an interstitial matrix (IM). The main components of vascular BMs include type IV collagen, laminin and nidogen. Minor components include type XV collagen and typ XVIII collagen. The components of the BM self-assemble into sheet-like structures. The BM is tightly connected to the IM through interactions between collagen type I and VI and collagen type IV and XV.

FIG. 1B. Isoforms of Type XVIII collagen. Collagen type XVIII exists in three isoforms, which differ in their N-terminus. The COL18A1 gene encodes these variants by the use of two promoters and alternative splicing. The short isoform has a different signal peptide from the other two, and is coded by promoter 1, while the others have the same signal peptide and are both coded by promoter 2. All isoforms include a thrombospondin-like domain, heparin sulphate chains and a globular C-terminal containing the type XVIII collagen fragment, endostatin.

FIG. 2. COI-18N antibody specific reactivity against type XVIII collagen. Monoclonal NB632-13H11/G5 antibody reaction towards standard peptide, truncated, eleongated and de-selection peptides is shown. The antibody has high reactivity towards standard peptide and no or minimal cross-reactivity with the other peptides.

FIG. 3. COL-18N correlation with ABR. Serum from 35 male HF patients aged 26 and over was measured with the COI-18N ELISA. Correlations between vascular endothelial type XVIII collagen concentration and ABR were analyzed using Spearman rank correlation coefficient and shown r=0.45, p<0.006. Differences between ABR and COL-18N levels were considered statistically significant if p<0.05 and significant levels are displayed as: *=p<0.01, and ***=p<0.001.

### DEFINITIONS

As used herein, the term “N-terminal epitope” refers to an N-terminal peptide sequence at the extremity of a polypeptide, i.e., at the N-terminal end of the polypeptide, and is not to be construed as meaning in the general direction thereof.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain conain the variable region, while the C-terminal portions of each of the heavy and light chains make up the constant reion. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

As used herein, the term “competitive ELISA” refers to a competitive enzyme-linked immunosorbent assay and is a technique known to the person skilled in the art.

As used herein, the term “sandwich immunoassay” refers to the use of at least two antibodies for the detection of an antigen in a sample, and is a technique known to the person skilled in the art.

As used herein, the term “short isoform of collagen type XVIII” refers to the isoform of collagen type XVIII generated by promoter 1 wherein the N-terminal non-collagenous region includes the thrombospondin-1 like domain (TSP-1), but does not contain the Domain of the Unknown Function (DUF) or the Frizzled Domain (FZ). The term “intermediate isoform of collagen type XVIII” refers to the alternatively spliced isoform of collagen type XVIII generated by promoter 2 wherein the N-terminal non-collagenous region includes the TsP-1 and DUF, but does not comprise the FZ. The term “long isoform of collagen type XVIII” refers to the alternatively spliced isoform of collagen type XVIII generated by promoter 2 wherein the N-terminal non-collagenous region includes the TSP-1, the DUF and the FZ.

As used herein, the term “COL-18N” is used as shorthand to describe the herein disclosed specific assay for the N-terminal sequence EPERISEEVG (SEQ ID NO: 1) of the short isoform of collagen type XVIII.

### Examples

The presently disclosed embodiments are described in the following Examples, which are set forth to aid in the understanding of the disclosure and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperatures, etc.) but some experimental errors and deviations should be accounted for. Unless indicated otherwise, parts are parts by weight, molecular weight is weight average molecular weight temperature is in degrees Centigrade, and pressure is at or near atmospheric.

### Patient Samples

Serum was collected from 35 male HF patients aged 26 and over. This cut-off age was chosen as collagen turnover wears off at the closure of the growth plate at the age of approximately 25 years (21). The patients had a treatment history of either on-demand medication upon bleeding episodes or intake of a low dosage of prophylaxis of 5-19 IU/kg recombinant FVIII, 203 times/week. Patients had varying degrees of HF arthropathy defined by the World Federation of Haemophilia Physical Examination Score (Gilbert Score) and by radiologic evaluation according to the Pettersson score. The patients' average ABR was 18.1 ranging from 2-46. Exclusion criteria were bleeding disorders other than haemophilia, human immunodeficiency virus infection, chronic obstructive pulmonary disease, medical history of joint disease or liver fibrosis, and treatment with anti-inflammatory biologics or steroids. Study participants were enrolled at the Department of Haematology, Peking Union Medical College Hospital, Beijing, China. The study was approved by the Peking Union Medical College Hospital, Chinese Academy of Medical Sciences, Ethics Review Board, with the serial number S-720. Signed informed consent was obtained from all subjects.

### Monoclonal Antibody Development for COL-18N

A peptide corresponding to the first 10 amino acids of the N-terminal epitope of the short isoform of human type XVIII collagen α1 chain (excluding signal peptide, ³⁴‘EPERISEEVG’⁴³; SEQ ID NO: 1)) was used to generate monoclonal neo-epitope specific antibodies. Beijing Administration Office of Laboratory Animal and animal ethics committee of NOrdic Bioscience approved the animal work. Generation of monoclonal antibodies was initiated by subcutaneous immunization of 6-8 week old Balb/C mice using 200 μL emulsified Freund's coplete adjuvant with 60 μg peptide conjugated to keyhole limpet hemocyanin (KLH). Consecutive immunizations were performed at 2-week intervals in Freund's incomplete adjuvant, until stable titer levels were reached. The mouse was boosted intravenously with 50 μg imunogen in 100 μL 0.9% sodium chloride solution and three days later the spleen cells were fused with SP2/0 myeloma cells (LGC Standards AB, Boras, Sweden) (21). They hybridomas were grown in 96-well plates and monoclonal growth was ensured by limited dilution. Clones were screened against the specific epitope (EPERISEEVG; SEQ ID NO: 1), elongated peptide (AEPERISEEVG; SEQ ID NO; 3) and truncated peptide (PERISEEVG; SEQ ID NO: 4). The mAb producing clone, NB632-13H11/G5, was selected based on reactivity to above-mentioned peptides, and antibody purified using Protein G columns (GE Healthcare, Hilleroed, Denmark).

The monoclonal antibody produced by the clone NB632-13H11/G5 was sequenced and the CDRs determined. The isotype of the mAb is mouse IgG2a/kappa, and the sequences of the chains of the mAb are as follows (CDRs underlined and in bold, constant region italic):

### Heavy Chain Amino Acid Sequence:

(SEQ ID NO: 20)
QVTLKESGPGILQPSQTLSLTCSFSGFSLSTSGMSVGWIRQPSGRG
LEWLAHIWWNDDKDYSPVLKSRLTISKDSSNNQVFLKIASVVAADT
ATYYCARMVFYTSTSGFDVWGAGTTVTVSSAKTTAPSVYPLAPVCG
DTTGSSVTLGCLVKGYFPEPVTLTWNSGSLSSGVHTFPAVLQSDLY
TLSSSVTVTSSTWPSQSITCNVAHPASSTKVDKKIEPRGPTIKPCP
PCKCPAPNLLGGPSVFIFPPKIKDVLMISLSPIVTCVVVDVSEDDP
DVQISWFVNNVEVHTAQTQTHREDYNSTLRVVSALPIQHQDWMSGK
EFKCKVNNKDLPAPIERTISKPKGSVRAPQVYVLPPPEEEMTKKQV
TLTCMVTDFMPEDIYVEWTNNGKTELNYKNTEPVLDSDGSYFMYSK
LRVEKKNWVERNSYSCSVVHEGLHNHHTTKSFSRTPGK

### Light Chain Amino Acid Sequence:

(SEQ ID NO: 21)
DVLMTQTPLSLPVSLGDQASISCRSSQNIVHTNGNTYLEWYLQKPG
QSPKLLIYRVSNRFSGVPDRFSGSGSGTDFTLKISRVEAEDLGVYY
CFHASHVPYTFGGGTKLEIKRADAAPTVSIFPPSSEQLTSGGASVV
CFLNNFYPKDINVKWKIDGSERQNGVLNSWTDQDSKDSTYSMSSTL
TLTKDEYERHNSYTCEATHKTSTSPIVKSFNRNEC

### COL-18N ELISA Protocol

The competitive COI-18N ELISA was performed as follows. A 96-well streptavidin-coated plate (Roche cat.: 19940279) was coated with 100 μl/well 1.25 ng/mL biotinylated synthetic peptide EPERISEEVG-K-Biotin (SEQ ID NO: 7) dissolved in coating buffer (20 mM Na₂HPO₄, 3.7 mM KH₂PO₄, 137 mM NaCl, 2.7 mM KCl, 0.1% Tween20, 1% BSA, pH7.4) and incubated 30 min at 20° C. The plate was washed five times with washing buffer (20 mM Tris, 50 mM NaCl, pH 7.2). 20 μL of the standard peptide (EPERISEEVG; SEQ ID NO: 6) or samples diluted in incubation buffer (20 mM Na₂HPO₄, 3.7 mM KH₂PO₄, 137 mM NaCl, 2.7 mM KCl, 0.1% Tween20, 1% BSA, 5% Liquid II pH7.4) were added to appropriate wells, followed by 100 μL/well monoclonal antibody NB632-13H11/G5, and incubated for 1 hr at 20° C. After washing, 100 μl rabbit-anti-mouse antibody (Jackson, 315-935-045) was added 1:3000 dissolved in coating buffer and incubated 1 hr at 20° C., 300 rpm. After final five times was, the wells were incubated with 100 μL tetramethylbenzidine (TMB) (Kem-En-Tec cat. 438OH) at 20° C., 300 rpm in the dark for 15 min., followed by the addition of 100 μL/well-stopping solution (1% H₂SO₄). The colorimetric reaction was measured at 450 nm with 650 nm as reference, and a calibration curve was plotted using a 4-parametric mathematical fit model.

### COL-18N Technical Evaluation

Technical assay validation was performed according to international guidelines. The lower limit of detection (LLOD) was calculated as mean+3× standard deviation (SD) determined from 21 zero samples (i.e., the assay buffer). The upper limit of detection (ULOD) was determined as the mean−3×SD of 10 measurements of standard A (1000 ng/ml). The lower limit of quantification (LLOQ) was determined by the lowest possible concentration with an imprecision of less than 30%. The intra- and inter-assay variations were calculated as the mean of the variation of seven human samples by 10 independent runs in duplicates. Dilution recovery was determined in a 2-fold dilution of two human serum and three human citrate plasma, calculated as percentage recovery of diluted matrices compared to undiluted ones. Spiking recovery was assessed in human serum and citrate plasma spiked with standard peptide at concentrations covering the entire measure range or by combining two samples of similar concentration in order to double the concentration. Spiking recovery was calculated as the measured amount percentage recovery of the theoretical amount. Interference by hemoglobin, lipemia, biotin, and human antibodies against moust antigens by human anti-mounse antibody (HAMA) was determined by adding two-fold dilutions to a serum sample of known concentration. Concentrations started at 0.500 mmol/I hemoglobin, 0.56 mmol/I lipemia, 160 μg/l biotin and 2010 ng/ml HAMA. Recovery percentage was calculated with the normal serum sample as a reference value. Analyte stability was determined for two healthy human serum samples and one healthy citrate plasma sample for four freeze-thaw cycles and calculated as the percentage recovery of the first freeze-thaw cycle. Same samples were tested at 2 hrs, 4 hrs and 24 hrs at 4° C. and 20° C. against non-stressed analytes. Finally, antibody specificity was assessed by a sanity check testing reactivity towards standard (EPERISEEVG; SEQ ID NO: 1), elongated (AEPERISEEVG; SEQ ID NO: 3), truncated (PERISEEVG; SEQ ID NO: 4) and de-selection peptides EPQIDEKKK; SEQ ID NO: 8) and CPERALERR (SEQ ID NO: 9)).

### Statistics

Correlations between serum COL-18N concentration and ABR were analyzed using Spearman rank correlation coefficient with GraphPad Prism v6 (GraphPad Software, La Jolla, CA, USA). Differences were considered statistically significant if p<0.05.

### Results and Discussion

A novel competitive ELISA using a monoclonal antibody to detect COL-18N in human serum and plasma (citrate, EDT, heparin) samples was developed and evaluated.

The main findings were:
serum COL-18N levels were correlated with ABR in HF patients, and
a technically stable assay for detecting COL-18N in human serum and human plasma with acceptable intra-inter assay variations and acceptable dilution and spike recoveries.

### Characterization of COL-18N ELISA

A competitive COL-18N ELISA that can assess endothelial BM degradation was developed. The technical performance of the ELISA is summarized in table 1, providing a measurement range from 4.8-671 ng/ml, intra- and inter-variability at 7% and 13% respectively, dilution and spike recovery within 100±20%, and analytic stability with no immunoassay interference. The normal concentration of COL-18N in serum (16.6 ng/ml), plasma citrate (12.5 ng/ml), EDTA plasma (13.2 ng/ml), and heparin plasma (15.8 ng/ml) was consistent regardless of matrices.

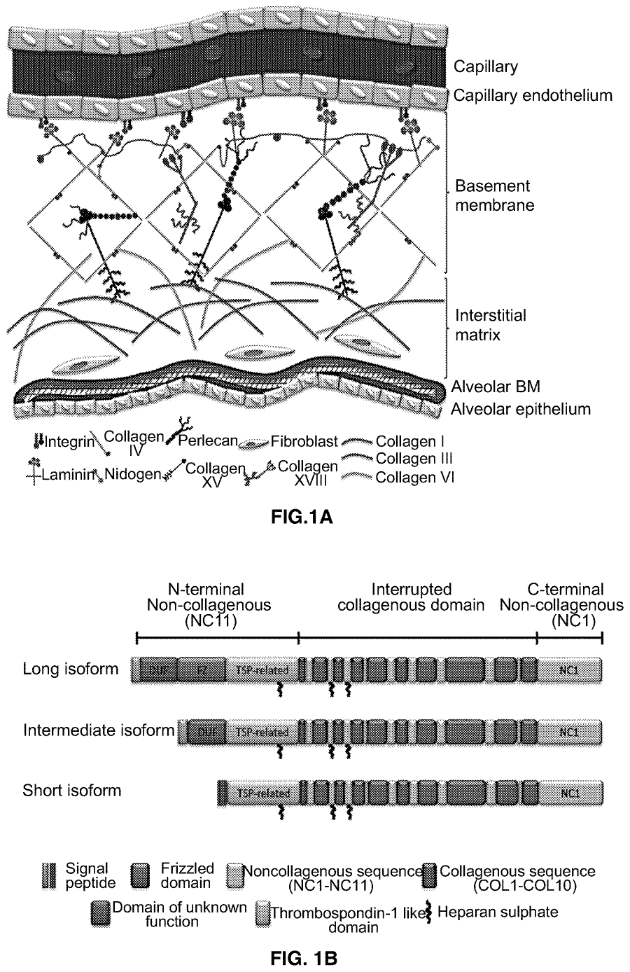

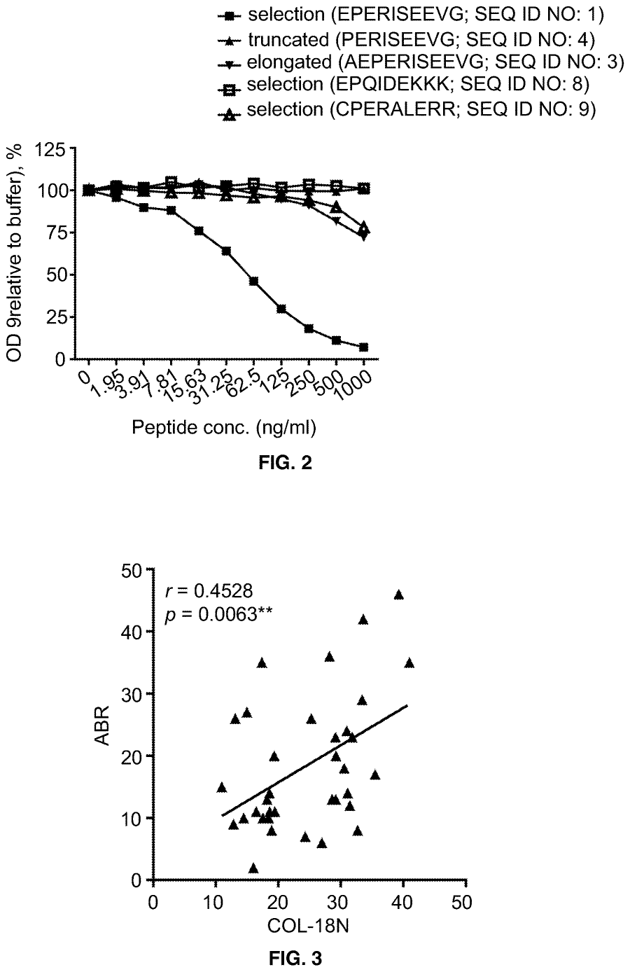

The NB632-13H11/G5 antibody specially recognized the first 10 amino acids of N-terminus type XVIII collagen α1 chain short isoform (selection) (FIG. 2). The antibody showed no or minimal reactivity towards related peptides, indicating a high specificaty (FIG. 2). Possible cross-reactivity with N-terminus of intermediary and long forms of collagen type XVIII is not plausible. The three isoforms of collagen type XVIII are encoded by the CL18A1 gene by the use of two different promoters and alternative splicing (18,19) (FIG. 1B). As a result N-terminus of the short isoform becomes entirely different from N-terminus of the other two isoforms.

The technical evaluation of the competitive COI-18N ELISA revealed a stable sensitive assay with high specificity towards the N-terminus of vascular form of type XVIII collagen including high accuracy and precision of the assay.

TABLE 1
Technical performance of COL-18N ELISA

| Measurements | Technical characteristics | |
|---|---|---|
| Lower limit of detection | 4.8 | ng/ml |
| Upper limit of detection | 671 | ng/ml |
| Lower limit of quantification | 7.3 | ng/ml |
| Intra-assay variability | 7% (accepted <10%) | |
| Inter-assay variability | 15% (accepted <15%) | |
| Dilution recovery | within 100 ± 20% | |
| Spiking recovery | within 100 ± 20% | |
| Freeze-thaw stability (4 cycles) | within 100 ± 20% | |
| Analyte stability | within 100 ± 20% | |
| (0-20 hrs at 4° C. and 20° C.) | | |
| Interference | No interference | |
| (Hgb, lipid, Biotin, HAMA) | | |
| Human healthy serum | 16.6 | ng/ml |
| (n = 10, mean value) | | |
| Human healthy plasma citrate | 12.5 | ng/ml |
| (n = 10, mean value) | | |
| Human healthy plasma EDTA | 13.2 | ng/ml |
| (n = 10, mean value) | | |
| Human healthy plasma heparin | 15.8 | ng/ml |
| (n = 10, mean value) | | |

COL-18N correlates to annual bleeding rate in HF patients. The haemorrhagic disorder haemophilia manifests clinically by repeated haemarthrosis resulting in unavoidable arthropathy in the absence of adequate treatment. One cardinal feature of medicinal intervention in haemophilia is lowering of ABR, albeit objective quantifiable parameters with high resolution are lacking.

The bleeding severity of haemophilia is generally inversely proportional to the degree of FVIII/IX activity in the plasma, although substantial variability in bleeding tendencies is well-known. Reduced spontaneous bleeding and lower requirements of factor concentrates are reported in a subset of 10-15% of severe HF patients (23,24). Also, development of inhibitors in non-severe HF patients may heighten the bleeding phenotype considerably (25).

Bleeding phenotype may be further compromised by large discrepancies amongst the FVIII assays caused by standardization of the assays (26) and may even be influenced by the type of FVIII concentrates used during therapy (27,28). Other assays, like thrombin generation, correlates to the bleeding phenotype in HF patients (29), but is inconsistent in HF patients with FVIII inhibitors despite the occurrence of thrombin generation (30).

In haemophilia, consequent to i) endothelial cell damage ii) bleeding and iii) delayed clotting and wound healing, the endothelial remodelling contributing to clinical symptoms of haemophilia and pathophysiological disease representation may be affected. It has now been found using the herein described assay that vascular endothelial type XVIII collagen correlates with ABR in HF patients (FIG. 3, r=0.45, p<0.006). Objective biomarkers of pathological processes, like those of degraded type XVIII collagen that associates with ABR, may assist in benchmarking treatments, monitoring patients and consequently may assist in drug development for the benefit of patients.

### CONCLUSION

In summary, the data combined suggests that the technically robust COL-18N biomarker can be related to pathologies involving vascular BM degradation and remodelling, which affects degradation of the short isoform of type XVIII collagen. In addition, the data enables the COL-18N biomarker to evaluate ABR for optimal treatment and monitoring of patients to prevent the development of arthropathy.

In this specification, unless expressly otherwise indicated, the word “or” is used in the sense of an operator that returns a true value when either or both of the stated conditions is met, as opposed to the operator ‘exclusive or’ which requires that only one of the conditions is met. The ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge at the date hereof.

### REFERENCES

1. Funk et al. Haemophilia 2002; 8:98-103.
2. Fischer et al. Blood 2013; 122:1129-36.
3. Gringeri et al. J Throm Haemost 2011; 9:700-10.
4. Lundin et al. Haemophilia 2005; 11:116-22.
5. Tomono et al. Cell Struct Funct 2002; 27:9-20.
6. Ricard-Blum S, Ruggiero F, Pathol Biol 2005; 53:430-42.
7. Manon-Jensen et al. J Throm Haemost 2016; 14:438-48.
8. Elamaa et al. Matrix Biol 2003; 22:427-42.
9. Saarela et al. Am J Pathol American Society for Investigative Pathology; 1998; 153:611-26.
10. Seppinen L, Pihlajaniemi T. Matrix Biol International Society of Matrix Biology; 2011; 30:83-92.
11. Marneros A G, Olsen B R. FASEB J 2005; 19:716-28.
12. Passos-Bueno et al. An Acad Bras Cienc 2006; 78:123-31.
3. Marneros et al. EMBO J 2004; 23:89-99.
14. Utriainen et al. Hum Mol Genet 2004; 13:2089-99.
15. Marneros A G, Olsen B R Investig Opthalmology Vis Sci 2003; 44:2367.
16. Fukai N. EMBO J 2002; 21:1535-44.
17. Aikio et al. Proc Natl Acad Sci USA 204; 111: E3043-52.
18. Carmeliet P, Collen D. Am J Physiol 1997; 273:2091-104.
19. Karsdal et al. Biomarkers 2013; 18:375-8.
20. Karsdal et al. Assay Drug Dev Technol 2013; 11:70-92.
21. Iki et al. Osteoporos Int 2004; 15:981-91.
22. Gefter et al. Somatic Cell Genet 1977; 3:231-6.
23. Aledort et al. J Intern Med 1994; 236:391-9.
24. Aznar et al. Haemophilia 2000; 6:170-6.
25. van Velzen et al. Thromb Haemost 2015; 115:543-50.
26. Barrowcliffe et al. Semin Thromb Hemost 2002; 28:247-56.
27. Mikaelsson M. Haemophilia 1998; 4.
28. Mikaelsson M, Oswaldsson U. Semin Throm Hemost 2002; 28:257-64.
29. Brummel-Ziedins et al. J Throm Haemost 2009; 7:825-32.
30. Ragni et al. Haemophilia 2016; 22:240-7.

1. An antibody specifically reactive with a short isoform of collagen type XVIII, wherein said antibody does not react with an intermediate isoform of collagen type XVIII or with a long isoform of collagen type XVIII.

2. The antibody of claim 1, wherein the antibody is specifically reactive with an N-terminal epitope of the short isoform of collagen type XVIII.

3. The antibody of claim 2, wherein the antibody is specifically reactive with the N-terminal epitope comprising an N-terminal amino acid sequence H₂N-EPERISEEVG (SEQ ID NO: 1).

4. The antibody of claim 2, wherein the antibody is specifically reactive with an N-terminal epitope comprising the N-terminal amino acid sequence H₂N-EPERIS (SEQ ID NO: 2).

5. The antibody of claim 3, wherein the antibody does not specifically recognise or bind an N-extended elongated version of said N-terminal amino acid sequence which is H₂N-AEPERISEEVG (SEQ ID NO: 3) and/or does not specifically recognise or bind an N-truncated version of said N-terminal amino acid sequence which is H₂N-PERISEEVG (SEQ ID NO: 4).

6. The antibody of claim 1, wherein the antibody is a monoclonal antibody.

7. A method of immunoassay for detecting or quantitating in a sample a short isoform of collagen type XVIII, wherein said method comprises contacting the sample of the short isoform of collagen type XVIII with an antibody specifically reactive with the short isoform of collagen type XVIII, wherein said antibody does not react with an intermediate isoform of collagen type XVIII or with a long isoform of collagen type XVIII, and determining an amount of binding of said antibody.

8. The method of claim 7 wherein the method comprises detecting or quantitating an N-terminal epitope of short isoform collagen type XVIII.

9. The method of claim 8, wherein the N-terminal epitope is comprised in the N-terminal amino acid sequence H₂N-EPERISEEVG (SEQ ID NO: 1).

10. The method of claim 8, wherein the N-terminal epitope is comprised in the N-terminal amino acid sequence H₂N-EPERIS (SEQ ID NO: 2).

11. The method of claim 7, wherein the sample is a biofluid.

12. The method of claim 11, wherein the biofluid is serum, plasma, urine, cerebrospinal fluid, or amniotic fluid.

13. The method of claim 7, wherein the immunoassay is a competition assay or a sandwich assay.

14. The method of claim 7, wherein the immunoassay is a radioimmunoassay or an enzyme-linked immunosorbent assay.

15. The method of claim 7, wherein the method further comprises correlating a quantity of said short isoform collagen type XVIII determined by said method with standard haemophilic disease samples of known disease severity to evaluate a severity of a haemophilic disease.

16. The method of claim 7, wherein the method further comprises comparing a quantity of said short isoform collagen type XVIII determined by said method with standard values associated with healthy subjects to evaluate a presence and/or a severity of a haemophilic disease.

17. The method of claim 7, wherein the method further comprises quantifying the amount of collagen type XVIII in at least two samples obtained from a subject at a first time point and at at least one subsequent time point,
wherein an increase in the quantity of collagen type XVIII from the first time point to the at least one subsequent time point is indicative of a deterioration in a haemophilic disease from the first time point to the at least one subsequent time point, or
wherein a decrease in the quantity of collagen type XVIII from the first time point to the at least one subsequent time point is indicative of an improvement in a haemophilic disease from the first time point to the at least one subsequent time point.

18. The method of claim 15, wherein the haemophilic disease is haemophilic arthropathy.

19. A method for evaluating the efficacy of a drug for treating a haemophilic disease, wherein the method comprises using a method of immunoassay for quantitating in a sample comprising a short isoform collagen type XVIII an amount of collagen type XVIII in at least two samples obtained from a subject at a first time point and at at least one subsequent time point during a period of administration of the drug to the subject,
wherein said method of immunoassay comprises contacting the sample comprising said short isoform collagen type XVIII with an antibody specifically reactive with the short isoform collagen type XVIII,
wherein said antibody does not react with an intermediate isoform of collagen type XVIII or with a long isoform of collagen type XVIII, and determining an amount of binding of said antibody, and
wherein a reduction in the amount of collagen type XVIII from the first time point to the at least one subsequent time point during the period of administration of the drug is indicative of an efficacious drug for treating a haemophilic disease.

20. An assay kit for determining the quantity of short isoform collagen type XVIII, the kit comprising an antibody specifically reactive with short isoform collagen type XVIII, wherein said antibody does not react with intermediate isoform collagen type XVIII or with long isoform collagen type XVIII, and at least one of:
a streptavidin coated 96 well plate;
a peptide which is reactive with said antibody, which may be a biotinylated peptide H₂N-EPERISEEVG-L-Biotin (SEQ ID NO: 5), wherein L is an optional linker;
an optionally biotinylated secondary antibody for use in a sandwich immunoassay;
a calibrator peptide comprising the N-terminal sequence H₂N-EPERISEEVG (SEQ ID NO: 6);
an antibody HRP labeling kit;
an antibody radiolabeling kit; or
an assay visualization kit.


SEQUENCE LISTING (21 sequences)

SEQ ID NO: 1 - protein, 10 aa, synthetic construct
EPERISEEVG

SEQ ID NO: 2 - protein, 6 aa, synthetic construct
EPERIS

SEQ ID NO: 3 - protein, 11 aa, synthetic construct
AEPERISEEVG

SEQ ID NO: 4 - protein, 9 aa, synthetic construct
PERISEEVG

SEQ ID NO: 5 - protein, 10 aa, synthetic construct
  SITE 10: note = C-terminus biotinylated with optional linker between C-terminus of peptide and biotin residue
EPERISEEVG

SEQ ID NO: 6 - protein, 10 aa, synthetic construct
EPERISEEVG

SEQ ID NO: 7 - protein, 11 aa, synthetic construct
  SITE 11: note = C-terminus biotinylated
EPERISEEVGK

SEQ ID NO: 8 - protein, 9 aa, synthetic construct
EPQIDEKKK

SEQ ID NO: 9 - protein, 9 aa, synthetic construct
CPERALERR

SEQ ID NO: 10 - protein, 7 aa, synthetic construct
TSGMSVG

SEQ ID NO: 11 - protein, 16 aa, synthetic construct
HIWWNDDKDYSPVLKS

SEQ ID NO: 12 - protein, 12 aa, synthetic construct
MVFYTSTSGFDV

SEQ ID NO: 13 - protein, 16 aa, synthetic construct
RSSQNIVHTNGNTYLE

SEQ ID NO: 14 - protein, 7 aa, synthetic construct
RVSNRFS

SEQ ID NO: 15 - protein, 9 aa, synthetic construct
FHASHVPYT

SEQ ID NO: 16 - protein, 79 aa, synthetic construct
RSSQNIVHTNGNTYLEWYLQKPGQSPKLLIYRVSNRFSGVPDRFSGSGSGTDFTLKISRVEAEDLGVYYCFHASHVPYT

SEQ ID NO: 17 - protein, 81 aa, synthetic construct
TSGMSVGWIRQPSGRGLEWLAHIWWNDDKDYSPVLKSRLTISKDSSNNQVFLKIASVVAADTATYYCARMVFYTSTSGFDV

SEQ ID NO: 18 - protein, 112 aa, synthetic construct
DVLMTQTPLSLPVSLGDQASISCRSSQNIVHTNGNTYLEWYLQKPGQSPKLLIYRVSNRFSGVPDRFSGSGSGTDFTLKISRVEAEDLGVYYCFHASHVPYTFGGGTKLEIK

SEQ ID NO: 19 - protein, 122 aa, synthetic construct
QVTLKESGPGILQPSQTLSLTCSFSGFSLSTSGMSVGWIRQPSGRGLEWLAHIWWNDDKDYSPVLKSRLTISKDSSNNQVFLKIASVVAADTATYYCARMVFYTSTSGFDVWGAGTTVTVSS

SEQ ID NO: 20 - protein, 452 aa, synthetic construct
QVTLKESGPGILQPSQTLSLTCSFSGFSLSTSGMSVGWIRQPSGRGLEWLAHIWWNDDKDYSPVLKSRLTISKDSSNNQVFLKIASVVAADTATYYCARMVFYTSTSGFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - protein, 219 aa, synthetic construct
DVLMTQTPLSLPVSLGDQASISCRSSQNIVHTNGNTYLEWYLQKPGQSPKLLIYRVSNRFSGVPDRFSGSGSGTDFTLKISRVEAEDLGVYYCFHASHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC